(19) United States
(12) Patent Application Publication
(10) Pub. No.: US 2009/0093849 A1
(43) Pub. Date: Apr. 9, 2009

(54) METATARSAL FIXATION SYSTEM

(76) Inventor: Greg Grabowski, N. Miami Beach, FL (US)

Correspondence Address: Timothy H. Van Dyke;Beusse Wolter Sanks Mora & Maire, Suite 2500, 390 North Orange Avenue, Orlando, FL, 32801, US

(21) Appl. No.: 11/866,703

(22) Filed: Oct. 3, 2007

(51) Int. Cl. A61B 17/70; A61B 17/56; A61B 17/04; A61B 17/58

(52) U.S. Cl. 606/280; 606/286; 606/301

(57) ABSTRACT

Disclosed herein is a fixation system comprising a plate configured for fixation of one or more bones of the foot. The system utilizes at least two sets of screws which interconnect upon fixation of the plate. The two sets of screws cross several different planes of cortical bone tissue, which serves to increase stability of the fixation system. Also disclosed are methods of using the fixation system.

### BACKGROUND

The metatarsals as a unit in the forefoot provide a broad plantar (on the sole) surface for load sharing. They are mobile in the sagittal plane (up and down) and individually the metatarsal heads can alter position to cope with uneven ground. This even sharing of weight also protects the skin from injury. Metatarsal fractures are usually caused by the blow of a heavy object dropped onto the forefoot or by a twisting injury. Fractures of the shaft can be caused by twisting of the body with the toes fixed, applying torque to the foot. Avulsion (pull-off) fractures occur particularly at the base of the 5th metatarsal.

The first, second and fifth metatarsals are more commonly injured than the others. The first metatarsal is shorter and wider than the other metatarsals. It also has a lack of interconnecting ligaments between itself and the second metatarsal. This allows for independent motion. The head of the first metatarsal is thought to bear one third of body weight. Three types of fracture predominate: avulsion, proximal shaft and mid shaft. Any evidence of instability of the fracture requires operative fixation. Traditionally, fixation can take the form of a simple lag screw, plate (across the cuneiform) or, in the case of a comminuted fracture, an external fixator device.

One surgical treatment, known as open reduction and internal fixation, usually requires that pins, wires and/or screws be inserted to stabilize the bones and joints and hold them in place until healing is complete. This treatment protocol re-establishes the normal anatomy of the mid-foot while the fractured bones mend. In one typical procedure, a pin or screw is introduced medially into the internal cuneiform and through the base of the second metatarsal bone.

In some cases, fusion of the joint between the first and second metatarsals and the middle and/or internal cuneiforms may be necessary. Arthrodesis may be indicated where arthritis arises in patients with a prior Lisfranc or similar injury, or where an acute fracture/dislocation has occurred anywhere at the mid-foot.

The use of pins, staples or screws is often acceptable for younger patients, especially where the injury is not too severe. However, this form of fixation frequently results in non-union in mid-foot arthrodesis attempts, possibly because the bone fragments and/or joints cannot be sufficiently immobilized by pins, screws or staples alone. Consequently, there is a significant need for a fixation device that provides solid fixation and stabilization of a mid-foot injury. Broad treatment possibilities also require that the fixation device be capable of multiple points of attachment to the mid-foot bones and bone fragments.

### SUMMARY

The subject invention relates to the discovery that improved fixation of metatarsal bones following injury is a desired objective. The invention provides a metatarsal fixation system that has a remarkable increase in stability of the injured site. One embodiment of the present fixation system combines a unique configuration of screws, which allows for securement into cortical bone at three different planes. In a particular embodiment, the system comprises a fixator plate having an elongated body portion with several apertures defined therein. A first set of screws are designed for placement through the apertures of the fixator plate, which act to secure the plate by traversing a first plane of cortical bone. The first set of screws comprises at least one screw through which a cross-passage is defined. A second set of screws is designed for positioning through the cross-passages of the first set of screws. Accordingly, upon securement of the fixator plate to the bone of a patient using the first set of screws, the second set of screws are positioned through the cross-passages of the secured first set of screws. The second set of screws transverses a second and third plane of cortical bone both separate from the first plane of cortical bone traversed by first set of screws. The interconnecting of the first and second set of screws results, in this embodiment, in the traversing of at least three different cortical planes of bone, thereby providing a dramatic improvement in stability of the fixator plate.

Those skilled in the art will appreciate that in alternative embodiments, the interconnection of the first and second set of screws may or may not transverse three different planes of cortical bone, but three different planes of bone nonetheless.

Naturally, the type of bone that the sets of screws will transverse will be dependent on the type of injury and location of the fixation system.

### BRIEF DESCRIPTION OF THE DRAWINGS

FIG. 1 shows a side view of one screw embodiment.

FIG. 2 shows a side view of another screw embodiment.

FIG. 3 shows a plate washer embodiment sideview (a) and front view (b).

FIG. 4 shows a top view of a fixator plate embodiment.

FIG. 5 shows a cross-sectional view of a fixation system embodiment fixated to the foot of a patient.

FIG. 6 shows a bottom view of a fixation system embodiment fixated to the foot of the patient. The view is transparent showing the interlocking of the screws.

FIG. 7 shows a drill guide embodiment. FIG. 7a shows a side view of the embodiment and FIG. 7b shows a perspective view of the embodiment. FIG. 7c shows a side view of the drill guide embodiment in use. FIGS. 7d-e show an example of a base screw utilized with the drill guide embodiment.

### DESCRIPTION OF ILLUSTRATIVE EMBODIMENTS

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

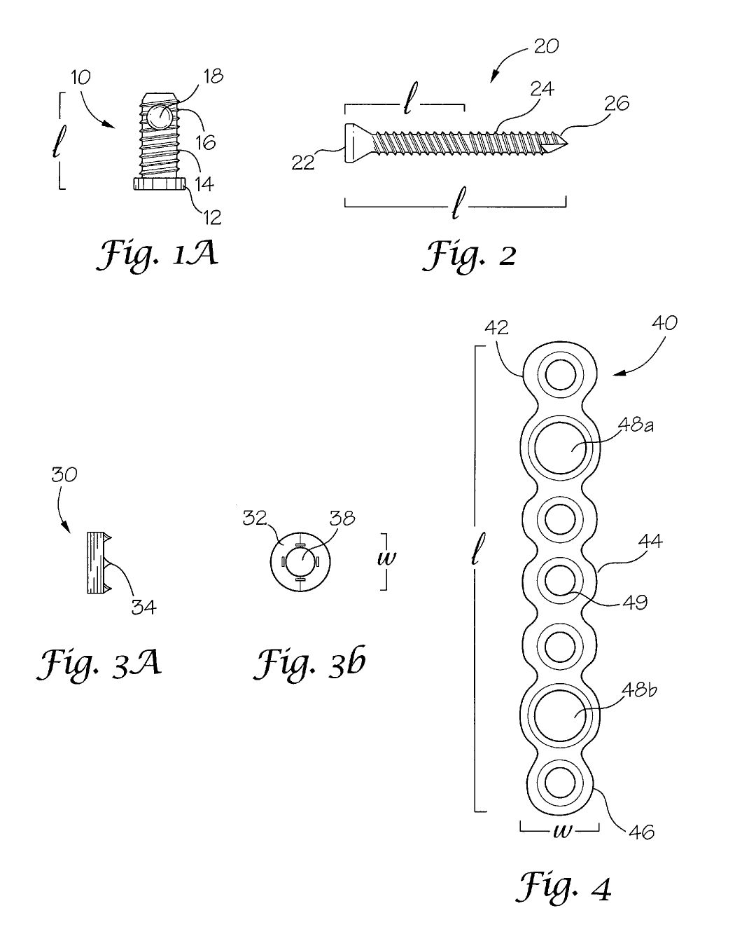

Turning to FIG. 1, a side view of a base screw 10 is shown. The base screw 10 comprises a proximal head end 12 and a distal end 16 with an elongated body 14 spanning between respective ends. Defined at a location along the elongated body 14 is a cross-passage 18 defined in the elongated body 14.

FIG. 2 shows a side view of a cross-screw 20 comprising a proximal head end 22 and a distal end 26. The cross-screw comprises an elongated body spanning from respective ends. FIG. 3a-b shows a plate washer 30. The plate washer 30 comprises an arcuate structural component 32 with a hole 38 defined therein. Extending from the plane of the plane of the arcuate structural component 32 are protrusions 34 which assist inhibiting the cross-screw from rotating upon positioning.

FIG. 4 shows a top view of a fixator plate embodiment 40. The fixator plate 40 has a top end 42 and a bottom end 46 with an elongated body 44 spanning between respective ends. The fixator plate 40 also has a series of large 48a-b and small 49 apertures defined therein. The small apertures 49 allow for the surgeon to apply other fixators at a location appropriate according to the surgeon's judgment. The fixator plate 40 may be contoured or substantially flat depending on the anatomy and orthopedic problems specific to a given patient. The length l of the fixator plate may be any length suitable depending on the patient's anatomy. In most cases, the fixator plate is 50-150 mm long and 5-15 mm wide.

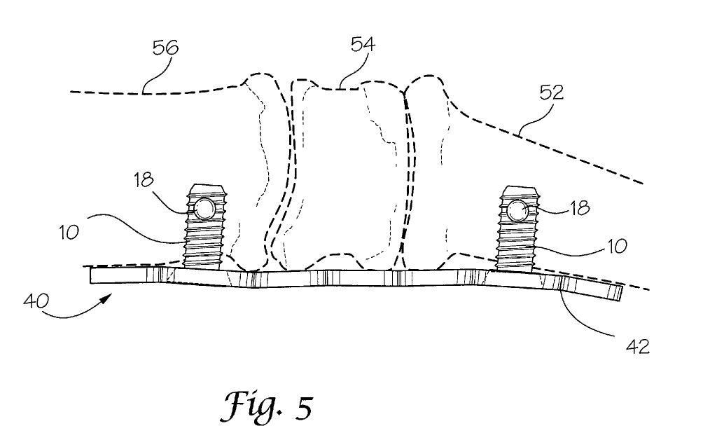

FIG. 5 shows a cross-section view of a patient's foot illustrating the implementation of a fixation system embodiment. In this embodiment, the fixator plate is positioned under the foot such that the top end 42 extends over the first metatarsal 52 and the bottom end 46 extends over the navicular 56. A portion of the elongated body 44 of the fixator plate 40 extends over the first cuniform 54. Shown in FIG. 5 are two base screws 10 which are put through large apertures 48a-b shown in FIG. 4 and secured to the first metatarsal 52 and the navicular 56.

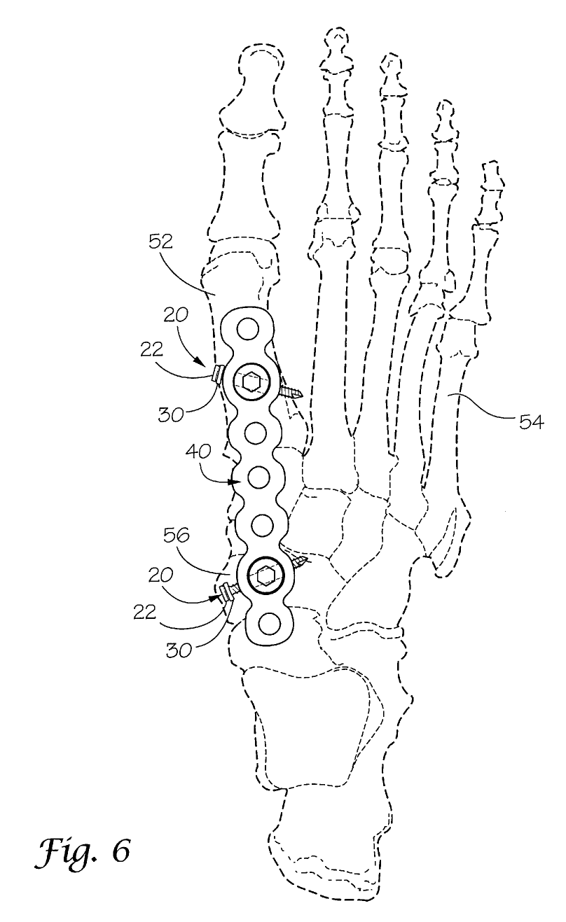

FIG. 6 shows a top transparent view of a patient's foot showing the implementation of the cross-screws 20. The fixator plate 40 is secured to the first metatarsal 52 and navicular 56 as shown in FIG. 5. The cross-screws 20 are positioned through the holes 18 (FIG. 5). Between the proximal head end 22 of the cross-screw 20 rests the plate washer 30 to help prevent backing out of the cross-screw 20. As can be seen from FIGS. 5 and 6, the fixation of the plate 40 involves the transversal of at least three planes of cortical bone tissue. The base screw 10 transverses one cortical bone plane and the cross-screws 20 transverse at least two cortical planes. Indeed, in many cases the cross-screw 20 passing through the base screw 10 which is secured to the navicular 56 passes through the two cortical planes of the navicular 56 and a cortical plane of the third cuniform 59.

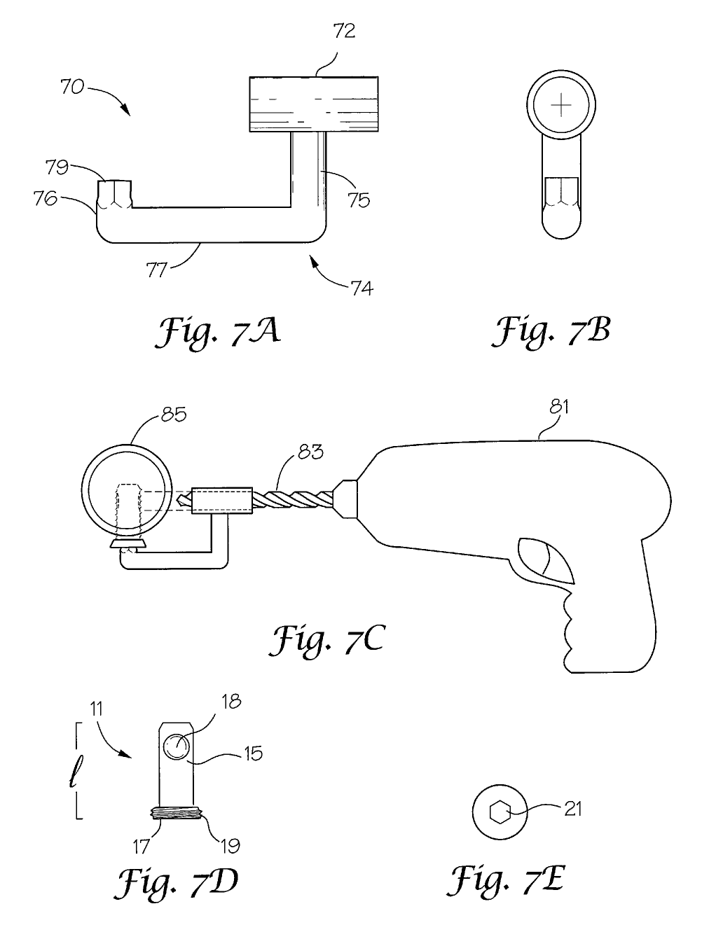

Turning to FIG. 7, a drill guide embodiment 70 is shown comprising a conduit 72 comprising a channel 73 into which a drill bit may be inserted. The conduit 72 is attached to or integral with a spacing member 74. The spacing member 74 comprises a screw-contact end 76 configured for contacting and engaging a base screw 10. The spacing member 74 may be of any configuration that is suitable for alignment of the drill guide to facilitate a drill bit to enter the cross-passage 18 of the base screw 10. In the embodiment shown in FIG. 7, the spacing member 74 comprises a first elongated portion 75 and a second elongated portion 77. The first elongated portion 75 is attached to or integral with the conduit 72 at a proximal end and is attached to or integral with the second elongated portion at a distal end. The second elongated portion 77 is attached to or integral with the first elongated portion 75 at a proximal end and is attached to or integral with the screw-contact end 76 at a distal end. The screw-contact end 76 comprises a hex-shaped extension 79 that is insertable into the base screw 10. The hex-shaped extension 79 is aligned so that when the base screw 11 is placed thereon, the cross-passage 18 of the base screw 11 is facing the conduit 72 of the drill guide 70. The drill guide 70 is spaced and configured to assist a user in accurately pinpointing a drill bit 83 of a drill 81 through a patients bone 85 and directly into the cross-passage 18 of the base screw 11.

The base screw 11 is an alternative embodiment to the base screw 10 discussed above. It comprises no threads on the elongated body 15, but comprises threads 19 on the proximal end 17. The threads 19 are provided to enable the base screw 11 to be screwed onto reciprocal threads provided within an aperture of the base plate. As shown in FIG. 7e, the base screw comprises a hex 21 defined in the proximal end of the 17 of the base screw 11. Those skilled in the art will appreciate that numerous configurations of the screw-contact end may be implemented with the key feature that when the base screw rests on the screw-contact end 76, the cross-passage 18 faces the conduit 72.

In most cases, the components of system embodiments described herein are made of metal. The components of the system embodiments described herein made be made of materials known in the art suitable for implants including but, not limited to, allograft, xenograft, or autograft material, metals, alloys, ceramics, polymers, and composites, or a combination thereof. In specific embodiments, the components are made of titanium alloy.

### EXAMPLE

### Surgical Procedure for Medial Arch Reconstruction

An exemplary surgical procedure summary is provided in this example.

Soft Tissue Dissection: Attention is first directed to the medial aspect of the foot. A curvilinear incision is placed on the medial aspect of the foot extending from the “Talus-Navicular (T-N)” joint distally to just past the “1^{st} Metatarsal-Medial Cuneiform (1^{st} M-C)” joint. Incision is made on the medial aspect of the foot on the junction between the plantar and dorsal skin surfaces. As dissection is taken though the subcutaneous tissue, careful attention is paid to preserve any neurovascular structures which pass through the operative site. Soft tissue dissection is taken down to the medial joint surfaces, exposing the bony surfaces of the Navicular, Medial Cuneiform, and 1^{st} Metatarsal bones both medially and plantarly. Plantar dissection will need to be adequate enough to accommodate the placement of the plantar plate. Note the Posterior Tibial Tendon may need to be reflected proximally.

Bony Procedures: Upon completion of soft tissue dissection, attention is the paid to the T-N and 1^{st} M-C joints. The joints surfaces are resected down the health subchondral bone. With the subchondral bone resected, corrective osteotomies can be performed at this time. Once bony correction has been achieved K-wires can be placed acting as temporary fixation, holding the bony alignment.

Plantar Plating: A measurement can be taken to determine the size of the plate desired. Once determined, the plate can be pre-bent to the approximate contour of the bony plantar surface. The plate is then introduced to the plantar surface. The plate can then be clamped into position temporally, and adequate positioning can be confirmed. With temporary fixation in place, a standard (non locking) screw can be placed into the most proximal screw position in the Navicular, followed by the most distal screw position in the 1^{st} Metatarsal. A central screw can be placed in one of the central holes further contouring the plate, if desired. With the two or more screws in position the plate clamp can be removed. The guide holes for the “base screws” can then be drilled, to the desired depth for the desired base screw length. The base screws are then placed into the guide holes and locked into the plate. Placing the axis guide into the “base screw,” the crossing screw position is then determined. Depending on the positioning of the cross screws, percutaneous application maybe required. The guide hole is drilled through the near cortex, through the base screw then through the far cortex of the bone. A measurement is then taken and the appropriate cross screw is then placed. If an acute angle is present on the cross screw—near cortex interface then a washer maybe required. With the “tricortical (base screw plus bicortical)” screws in position, the remaining holes in the plate can then be filled with desired screws. These screws can be locking as well as non-locking, and the original non locking screws placed prior to the base screws can also be exchanged if desired. With permanent fixation in place the K-wires used as temporary fixation can then be removed, these can be replaced with permanent screw fixation if desired. Closure is then performed in layers to the surgeon's preference.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

What is claimed is:

1. A bone fixation system comprising
a first set of fasteners each comprising a first fastener proximal end, a first fastener distal end and a first fastener elongated body spanning between said first fastener proximal and distal ends, wherein said first fastener elongated body comprises at least one cross-passage defined therein;
a fixator plate comprising a top end, a bottom end and a fixator plate elongated body spanning between said top and bottom ends, wherein said fixator plate elongated body comprises at least two fixator plate apertures defined therein;
a second set of fasteners each comprising a second fastener proximal end, a second fastener distal end and a second fastener elongated body spanning between said second fastener proximal and distal ends;
wherein at least one of said first set of elongated fasteners is transversely positioned through at least one of said at least two fixator plate apertures; and
wherein at least one of said second set of fasteners is transversely positioned through said at least one cross-passage.

2. The system of claim 1, wherein said fixator plate is 50-150 mm long and 5-15 mm wide.

3. The system of claim 1, wherein said fixator plate has a plurality of apertures aligned down the central axis of said fixator plate elongated body, said elongated body comprising at least two first fastener apertures sized and configured for accepting said first set of fasteners and at least one aperture comprising a size different from said at least two first fastener apertures.

4. The system of claim 1, wherein said first or second set of fasteners, or both, are made of a rigid material.

5. The system of claim 4, wherein said rigid material is metal, polymer, or bone.

6. The system of claim 5, wherein said first or second set of fasteners, or both are pins or screws.

7. The system of claim 6, wherein said proximal end of said first or second set of fasteners comprises a head portion wider than its respective elongated body.

8. The system of claim 6, wherein said second set of fasteners are screws, wherein the screws comprises a first thread pitch and a second thread pitch.

9. A bone fixation kit comprising
a first set of elongated fasteners each comprising a first fastener proximal end, a first fastener distal end and a first fastener elongated body spanning between said first fastener proximal and distal ends, wherein said first fastener elongated body comprises at least one cross-passage defined therein;
a fixator plate comprising a top end, a bottom end and a fixator plate elongated body spanning between said top and bottom ends, wherein said fixator plate elongated body comprises at least two apertures defined therein;
a second set of fasteners each comprising a second fastener proximal end, a second fastener distal end and a second fastener elongated body spanning between said second fastener proximal and distal ends; and
a drill guide comprising a conduit and a spacing member, said spacing member attached to or integrated with said conduit at a proximal end and engageable to said base screw at a distal end, said conduit and spacing member configured so as to guide a drill bit through said cross passage of said base screw when said base screw is placed through said fixator plate.

10. The kit of claim 9, wherein said fixator plate is 50-150 mm long and 5-15 mm wide.

11. The kit of claim 9, wherein said fixator plate has a plurality of apertures aligned down the central axis of said fixator plate elongated body, said elongated body comprising at least two first fastener apertures sized and configured for accepting said first set of fasteners and at least one aperture comprising a size different from said at least two first fastener apertures.

12. The kit of claim 9, wherein said first or second set of fasteners, or both, are made of a rigid material.

13. The kit of claim 12, wherein said rigid material is metal, polymer, or bone.

14. The kit of claim 12, wherein said first or second set of fasteners, or both, are pins or screws, or a combination thereof.

15. The kit of claim 9, wherein said proximal end of said first or second set of fasteners comprises a head portion wider than its respective elongated body.

16. The kit of claim 9, wherein said second set of fasteners are screws, wherein the screws comprises a first thread pitch and a second thread pitch.

17. The kit of claim 15, wherein said head portion of at least one of said first set of fasteners comprises a hex indentation defined therein.

18. The kit of claim 17, wherein said distal end of said spacing member comprises a hex-shaped member associable with said hex indentation.

19. The kit of claim 9, further comprising a plurality of washers.

20. A method of reconstructing a medial arch of a subject, said method comprising
obtaining a fixator plate comprising a top end, a bottom end and a fixator plate elongated body spanning between said top and bottom ends, wherein said fixator plate elongated body comprises a top fixator plate aperture and bottom fixator plate aperture defined therein;
securing said top end of said fixator plate to a first metatarsal of said subject by inserting a top first fastener through said top fixator plate aperture and into said first metatarsal, said top first fastener comprising a first fastener proximal end, a first fastener distal end, a first fastener elongated body spanning between said first fastener proximal and distal ends, and at least one cross-passage defined therein;
securing said bottom end of said fixator plate to a navicular of said subject by inserting a bottom first fastener through said bottom fixator plate aperture and into said navicular, said bottom first fastener comprising a first fastener proximal end, a first fastener distal end, a first fastener elongated body spanning between said first fastener proximal and distal ends, and at least one cross-passage defined therein;
inserting a top second fastener into said first metatarsal at a vector transverse to said top first fastener and through said at least one cross-passage of said top first fastener, said top second fastener comprising a second fastener proximal end, a second fastener distal end and a second fastener elongated body spanning between said second fastener proximal and distal ends; and
inserting a bottom second fastener into said navicular at a vector transverse to said bottom first fastener and through said at least one cross-passage of said bottom first fastener, said bottom second fastener comprising a second fastener proximal end, a second fastener distal end and a second fastener elongated body spanning between said second fastener proximal and distal ends.

21. The method of claim 20 said top first fastener traverses a plane of cortical bone of said first metatarsal of said patient and said bottom first fastener traverses a plane of cortical bone of said navicular of said patient.

22. The method of claim 20, wherein said top second fastener traverses a first and second plane of cortical bone of said first metatarsal and said bottom second fastener traverses a first and second plane of said navicular.

23. The method of claim 20, wherein said fixator plate comprises at least one auxiliary aperture additional to said top fixator plate aperture and said bottom fixator plate aperture.

24. The method of claim 20, wherein a fastener is inserted through said at least one auxiliary aperture and into a foot bone of said patient.

25. The method of claim 20, wherein said proximal end of said top and bottom second fasteners comprise a head portion wider than its respective elongated body, and the method further comprising putting a washer between said head portion of said top and bottom second fasteners and said first metatarsal and said navicular, respectively.
